(19) United States
(12) Patent Application Publication
(10) Pub. No.: US 2003/0233059 A1
(43) Pub. Date: Dec. 18, 2003

(54) Method and device for measuring myodynamia by cycle-type ergometer

(76) Inventors: Satoshi Nakayama, Osaka (JP); Naokata Ishii, Tokyo (JP); Chizuko Mishima, Osaka (JP)

Correspondence Address: Jonathan P. Osha, ROSENTHAL & OSHA L.L.P., 1221 McKinney Street, Suite 2800, Houston, TX, 77010, US

(21) Appl. No.: 10/405,054

(22) Filed: Apr. 1, 2003

(30) Foreign Application Priority Data
Apr. 1, 2002 (JP) 2002-099078

(51) Int. Cl. A61B 5/103

(52) U.S. Cl. 600/595

(57) ABSTRACT

The device has a torque measuring means for measuring the torque produced by pedals and a rotational speed measuring means for measuring the pedal rotational speed. Pedal load is controlled such that it increases gradually. The correlation between the torque achieved by a subject and the pedal rotational speed is measured at two or more points until the subject can no longer resist the pedal load. These measurement data are approximated by a curve. The device has a control means for estimating the maximum torque achievable by the subject to be the torque achieved by the subject as the pedal rotational speed approaches zero based on this measurement data curve. Thus, the device enables adequate evaluation of myodynamia without achieving maximum myodynamia.

### CROSS-REFERENCE TO RELATED APPLICATIONS

This application claims priority from Japanese Patent Application No. 2002-99078 filed Apr. 1, 2002.

### BACKGROUND OF INVENTION

1. Field of the Invention

This invention pertains to a method and device for measuring myodynamia by a cycle-type ergometer. More particularly, this invention pertains to techniques that make it possible to evaluate lower limb myodynamia without achieving maximum myodynamia.

2. Background Art

A conventional cycle-type ergometer (load exercise devices) comprises parts such as pedals, a pedal load device, a handle, a controller, a display, and a heart rate sensor.

The pedal load device is designed to control the pedal load to a specific level corresponding to a load set by the controller or to a load having a certain pattern, and to exercise feedback control such that the reading by the heart rate sensor reaches a heart rate set by the controller. The pedal load device is also designed to evaluate the endurance of the subject from the nature of fluctuation in heart rate.

Because the cycle-type ergometer evaluates by achieving maximum myodynamia, however, they lead to elevation of the heart rate and blood pressure of the subject, and cannot be used to evaluate myodynamia safely in the elderly.

This invention is developed upon considering on such problems, and addresses the problem of offering a method and device for measuring myodynamia by a cycle-type ergometer that can evaluate maximum myodynamia by a trial at, for example, 50% or less of the maximum myodynamia, minimize elevation of the heart rate and blood pressure of the subject, reduce the load on bones, and evaluate myodynamia safely even in the elderly.

### SUMMARY OF INVENTION

The invention is characterized in a cycle-type ergometer having a torque measuring

means for measuring the torque produced by pedal 20 and a rotational speed measuring means for measuring the pedal rotational speed, by having a control means for controlling the pedal load such that it gradually increases, measuring the correlation between the torque achieved by a subject and the pedal rotational speed at two or more points until the subject can no longer resist the pedal load, approximating these measurement data by a curve, and estimating the maximum torque achievable by the subject to be the torque achieved by the subject as the pedal rotational speed approaches zero based on this measurement data curve.

According to such a constitution, measurement data for achieved torque and pedal rotational speed are measured several times until the subject can no longer resist the pedal load and approximated by a curve, and the maximum torque achievable by the subject is estimated based on such a measurement data curve. Therefore, the maximum torque achievable by the subject can be known by a trial at, for example, 50% or less of the maximum myodynamia even without achieving maximum myodynamia, elevation of the heart rate and blood pressure of the subject can be minimized, the load on bones can be reduced, and myodynamia can be evaluated safely even in the elderly.

The invention is characterized by having a control means for detecting when the pedal rotational speed measured by the rotational speed measuring means reaches saturation when the subject is asked to pedal as hard as possible under a light load before raising the pedal load, and beginning to increase the pedal load based on this detection result.

According to such a constitution, by increasing the load after the rotational speed has become saturated, reliable and high-precision data can be sampled repeatedly, and it can be confirmed that the subject has reached the maximum speed at this load. The subject can see clearly the difference made by even a slight change in myodynamia due to the effect of daily training, and this becomes an incentive for daily training.

The invention is characterized by exercising control such that the pedal load increases in stages. According to such a constitution, it may be difficult to confirm whether or not the rotational speed is saturated when the pedal load is varied continuously, which can easily lead to errors in measurement data, but by increasing the load in stages, it can be observed when the rotational speed has become saturated, and the precision of measurement data can be improved.

The invention is characterized by controlling the increment increasing the load in stages to a lower increment if the pedal rotational speed measured by the above-mentioned rotational speed measuring means fell by a greater increment the last time and a greater increment if the pedal rotational speed fell by a lesser increment the last time when comparing the last time to the time before last, and keeping the range of fall in rotational speed within a specific range. According to such a constitution, the increment increasing the load in stages may be adjusted in various ways according to the myodynamic state of the subject, but when applying a load in stages and the resulting change in speed are observed without a technique for estimating the myodynamia of the subject at the start of pedaling, there may be too little adjustment in the increment increasing the load for persons who find the change in load too great and too much adjustment for persons who find the change in load too little. Therefore, this invention avoids the number of measurement data being too few for persons lacking strength resulting in a drop in myodynamia estimation precision, or measurement taking too long for persons with good strength resulting in a drop in myodynamia estimation precision due to fatigue.

The invention is characterized by having a pedal crank angle measuring means for measuring the crank angle of the pedal from a reference position during one revolution and the data of the subject to be sampled being the torque achieved at the same pedal crank angle during one revolution and the pedal rotational speed, and the invention is characterized by the data of the subject to be sampled being the torque achieved at the maximum torque during one revolution and the pedal rotational speed at this time. According to such a constitution, the timing for sampling data after the rotational speed becomes saturated is not whenever desired, and the torque during one revolution is varied. As a result, data can be sampled with consistent conditions and good precision by sampling data always at the same pedal crank angle or sampling data at the maximum torque during one revolution.

The invention is characterized by basing the curve approximating the measurement data on Hill formula. According to such a constitution, the approximation curve may have a certain degree of basis when finding the estimated maximum torque. The Hill formula expresses human myodynamic force and speed characteristics. Because torque is inversely correlated with myodynamia when the pedal crank angle is constant. The Hill formula is considered to have an effect improving precision when applied in this case. In addition, results of experiments by the present inventors were close to linear. Therefore, an easy linear approximation can reduce the number of measurement data, speed up measurement time by the device, and reduce the load on the subject.

The invention is characterized by estimating the maximum rotational speed as the speed when the load in the approximation curve reaches zero. According to such a constitution, the estimated maximum rotational speed is found for rotational speed in the same way as for myodynamia. Therefore, the subject can evaluate not only strength, but also quickness of action. This is the more important evaluation criterion for persons engaged in sports emphasizing speed more than strength.

The invention is characterized by creating a general correlation between age and maximum myodynamia statistically from the age and maximum achieved torque of many subjects and finding the maximum myodynamia of a new subject by referring to the muscle-torque age in the above-mentioned correlation chart, and the invention is characterized by creating a general correlation between age and maximum rotational speed statistically from the age and maximum rotational speed of many subjects and finding the maximum rotational speed of a new subject by referring to the rotational-speed age in the above-mentioned correlation chart. According to such a constitution, subjects who do not engage in sports can evaluate whether their own myodynamia or quickness is good or bad for their age when compared to other people, which can help raise their QOL (quality of life).

Furthermore, because it is not uncommon to find a correlation with physique or weight when comparing to other people, especially when comparing lower limb myodynamia, weight or physique may be normalized to a reference physique to eliminate these factors. Going further, evaluation reliability can be increased by normalizing body height or leg length to a reference.

If it is necessary to apply a load greater than the weight of the subject (especially for young people or athletes), this can cause the body to lift up, making it impossible to apply the load. For this reason, when the ergometer is made a recumbent type chair type, backrest 23 can be used to resist the load, making it possible to evaluate myodynamia without difficulty even when applying a load greater than the weight of the subject.

### BRIEF DESCRIPTION OF DRAWINGS

FIG. 1 shows a schematic side elevation of the device according to an embodiment of this invention for measuring myodynamia by a cycle-type ergometer.

FIG. 2 shows a block diagram of a controller for the same device.

FIG. 3 is a graph showing the correlation between brake force, rotational speed, and torque for the same device.

FIG. 4 is a graph illustrating an approximation curve based on a hill formula for finding the estimated maximum load and the estimated maximum rotational speed from data measured by the same device.

FIG. 5 shows a schematic side elevation of a recumbent type showing another embodiment.

### DETAILED DESCRIPTION

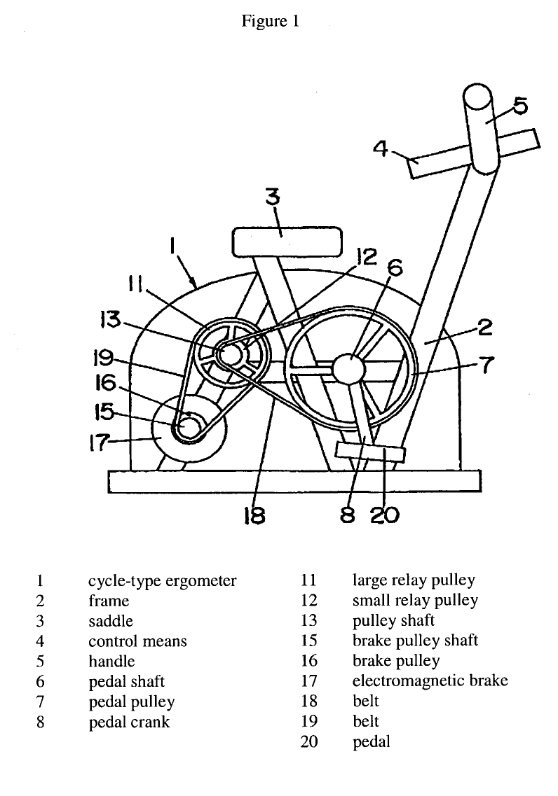

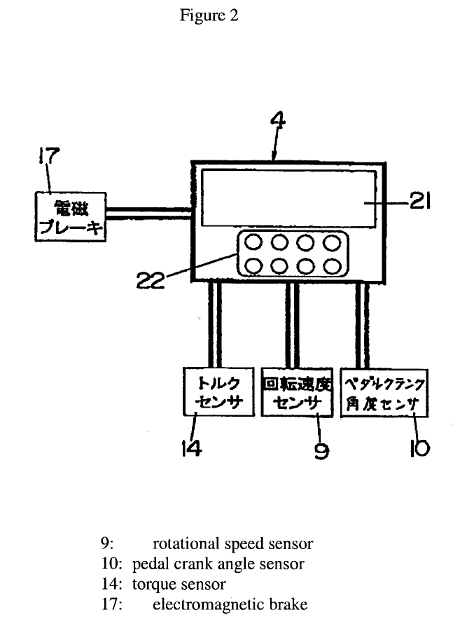

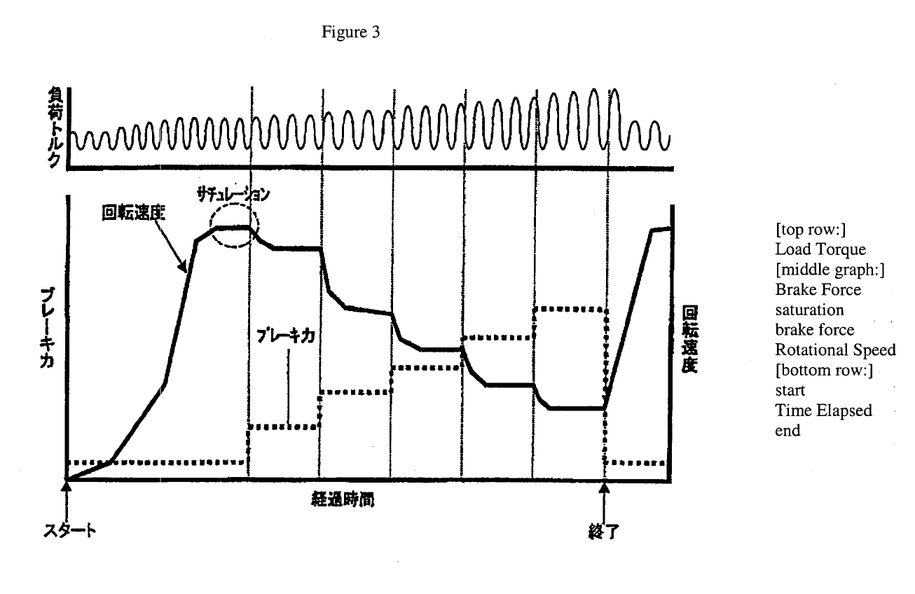

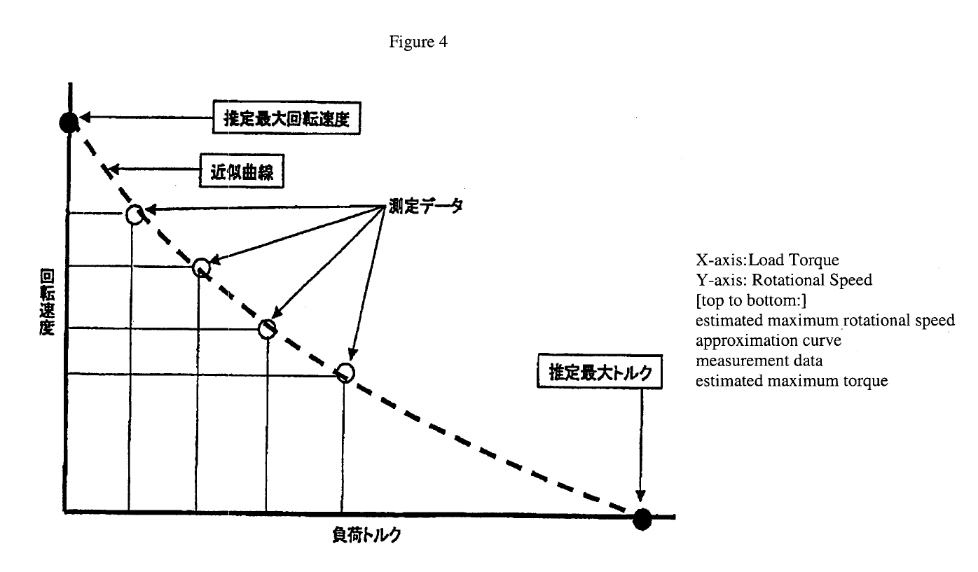

This invention will be explained in detail below based on a preferred embodiment. FIG. 1 shows a schematic side elevation of a device for measuring myodynamia by a cycle-type ergometer. FIG. 2 shows a block diagram of a controller. FIG. 3 is a graph showing the correlation between brake force, rotational speed, and torque. FIG. 4 is a graph illustrating an approximation curve for finding the estimated maximum load and the estimated maximum rotational speed from measured data.

A cycle-type ergometer 1 has a saddle 3, which is attached to a frame 2, and a handle 5, holding a controller 4 comprising a control means, which is also attached to frame 2. The saddle 3 and the handle 5 are designed such that they can be adjusted in height to match the build of the user.

A pedal shaft 6 is supported free to rotate on frame 2, and a pedal pulley 7 and a pedal crank 8 are provided to the pedal shaft 6. A rotational speed sensor 9 is connected to the pedal pulley 7, and designed to detect the rotational speed per unit time and input this to the controller 4. A crank angle sensor 10 is connected to the pedal crank 8, and designed to detect the angle of rotation of the pedal crank 8 from a reference position and input this to the controller 4. These sensors 9 and 10 are housed in the pedal shaft 6, but may also be installed in other locations.

A pulley shaft 13 is mounted on frame 2, and supports a large relay pulley 11 and a small relay pulley 12 free to rotate. A torque sensor 14 is housed in the pulley shaft 13, and designed to detect rotational torque and input this to the controller 4.

A brake pulley shaft 15 is mounted on frame 2, and a brake pulley 16 and an electromagnetic brake 17 are attached such that various high or low braking loads can be applied by a controlling electromagnetic brake 17. These are designed such that the load can be set by the controller 4.

Rotational force is transmitted by suspending a belt 18 between large the large-diameter pedal pulley 7 and the small-diameter relay pulley 12, and suspending a belt 19 between the large relay pulley 11 and the brake pulley 16.

Therefore, the load produced by the electromagnetic brake 17 is transmitted through the brake pulley 16, the belt 19, the large relay pulley 11, the small relay pulley 12, the belt 18, and the pedal pulley 7 to a pair of pedals 20 on the end of the pedal crank 8, and the user performs training by pedaling the pedals 20 against the resistance of this load.

The controller 4 has a display unit 21 and an operating unit 22 for inputting personal data and settings, and houses a memory and a computer unit (not shown).

Next, the method for using the cycle-type ergometer 1 will be explained. The user sits on the saddle 3, inputs the personal data of body height, weight, leg length, sex, and age into the controller 4, and starts measuring myodynamia. First, when the start measurement button is pressed, the controller 4 reduces the electromagnetic brake 17 to its lowest brake force. At this point, the user starts pedaling the pedals 20 as hard as possible, and the controller 4 does nothing while the speed is increasing based on the signal coming from the rotational speed sensor 9. When the speed coming from the rotational speed sensor 9 becomes saturated, the controller 4 stores the measurements for load torque and rotational speed at this time in the memory. Next, heavier braking is applied in stages by the electromagnetic brake 17, and the increment of speed relaxation in the measurements for load torque and rotational speed when rotational speed has subsided is stored in the controller 4. Next, even heavier braking is applied in stages, and the increment of speed relaxation in the measurements for load torque and rotational speed when rotational speed has subsided is stored in the controller 4. The increment of speed relaxation the first time and the increment of speed relaxation this time are compared, and if the increment of speed relaxation this time is greater, the increment of increase in brake force is reduced next time, and if it is less, the increment of increase in brake force is increased. Thus, brake force is varied in several stages, and the point when speed has decreased to 50% of the initial speed or lower is taken as the lowest brake force of the electromagnetic brake 17 and measurement is stopped. The timing of storing data after the speed has subsided may be when the pedal crank angle has become constant, or when the peak load torque is reached in one revolution.

To evaluate the myodynamia of the user from measurement data, first, the measurement data are plotted on a graph with torque on the horizontal axis and rotational speed on the vertical axis, and a curve passing through these points is approximated (see FIG. 4). This curve may be a hyperbolic curve or straight line represented by the Hill formula:

(P+a)(V+b)=(Po+a)b

P: load torque, V: rotational speed, Po: maximum torque, a, b: constants

The intersection between the approximation curve and the horizontal axis (load torque axis) is the estimated maximum torque of the user, and is used as a gauge for evaluating myodynamia. When the estimated maximum torque is divided by the length of pedal crank 8, this becomes the estimated maximum myodynamia. Because these gauges generally must be normalized before comparing between different individuals, the gauges are divided by body weight, for example, before comparing. In addition, because the angle at which the pedals 20 are pedaled for the pedal crank 8 of constant length differs depending on the length of the user's legs, the estimates may also be normalized by leg length, or if easier, by body height. The intersection between the approximation curve and the vertical axis (rotational speed axis) is the estimated maximum rotational speed, which is also used as a gauge for evaluating myodynamia.

Data for several hundred men and women of all ages were found using the method described above, the gauges were plotted on graphs by sex with actual age on the horizontal axis and gauge measurements on the vertical axis, and approximation curves statistically averaging the gauges for age were stored in advance in the controller 4 of the device of this invention for measuring myodynamia by the cycle-type ergometer 1. By this means, newly measured data can be matched to approximation curves averaging each gauge to find the corresponding age, and subjects can be described as young for their age if their actual age is less than the age found, and old for their age if their actual age is greater. This can be a great incentive for subjects to exercise.

As described in detail above, in this invention, measurement data for achieved torque and pedal rotational speed are measured several times until the subject can no longer resist the pedal load and approximated by a curve, and the maximum torque achievable by the subject is estimated based on such a measurement data curve. Therefore, the maximum torque achievable by the subject can be known by a trial at, for example, 50% or less of the maximum myodynamia even without achieving maximum myodynamia, elevation of the heart rate and blood pressure of the subject can be minimized, the load on bones can be reduced, and myodynamia can be evaluated safely even in the elderly.

In this device, the subject is asked to pedal as hard as possible and the load is increased after the rotational speed becomes saturated, reliable and high-precision data can be sampled repeatedly, and it can be confirmed that the subject has reached the maximum speed at this load. Therefore, the subject can see clearly the difference made by even a slight change in myodynamia due to the effect of daily training, and this becomes an incentive for daily training.

Now, it may be difficult to confirm whether or not the rotational speed is saturated when the pedal load is varied continuously, which can easily lead to errors in measurement data, but by increasing the load in stages, it can be observed when the rotational speed has become saturated, and the precision of measurement data can be improved.

Furthermore, the increment increasing the load in stages is controlled to a lower increment if the pedal rotational speed measured by the above-mentioned rotational speed measuring means fell by a greater increment the last time and a greater increment if the pedal rotational speed fell by a lesser increment the last time when comparing the last time to the time before last, and the range of fall in rotational speed is kept within a specific range. That is, the increment increasing the load in stages may be adjusted in various ways according to the myodynamic state of the subject, but when applying a load in stages and the resulting change in speed are observed without a technique for estimating the myodynamia of the subject at the start of pedaling, there may be too little adjustment in the increment increasing the load for persons who find the change in load too great and too much adjustment for persons who find the change in load too little. As a result, this avoids the number of measurement data being too few for persons lacking strength resulting in a drop in myodynamia estimation precision, or measurement taking too long for persons with good strength resulting in a drop in myodynamia estimation precision due to fatigue.

Next, the data of the subject to be sampled is the torque achieved at the same pedal crank angle during one revolution and the pedal rotational speed, or the torque achieved at the maximum torque during one revolution and the pedal rotational speed at this time. That is, the timing for sampling data after the rotational speed becomes saturated is not whenever desired, and the torque during one revolution is varied. As a result, data can be sampled with consistent conditions and good precision by sampling data always at the same pedal crank angle or sampling data at the maximum torque during one revolution.

The curve approximating the measurement data is based on the Hill formula as described above, and the maximum rotational speed is estimated as the speed when the load in the approximation curve reaches zero. Because the estimated maximum rotational speed is found for rotational speed in the same way as for myodynamia, the subject can evaluate not only strength, but also quickness of action. This has the advantage that this is the more important evaluation criterion for persons engaged in sports emphasizing speed more than strength.

A general correlation between age and maximum myodynamia is created statistically from the age and maximum achieved torque of many subjects, the maximum myodynamia of a new subject is found by referring to the muscle-torque age in the above-mentioned correlation chart, and the maximum rotational speed is found by referring to the rotational-speed age. As a result, subjects who do not engage in sports can evaluate whether their own myodynamia or quickness is good or bad for their age when compared to other people, which can help raise their QOL (quality of life).

Furthermore, because it is not uncommon to find a correlation with physique or weight when comparing to other people, especially when comparing lower limb myodynamia, weight or physique may be normalized to a reference physique to eliminate these factors. Going further, evaluation reliability can be increased by normalizing body height or leg length to a reference.

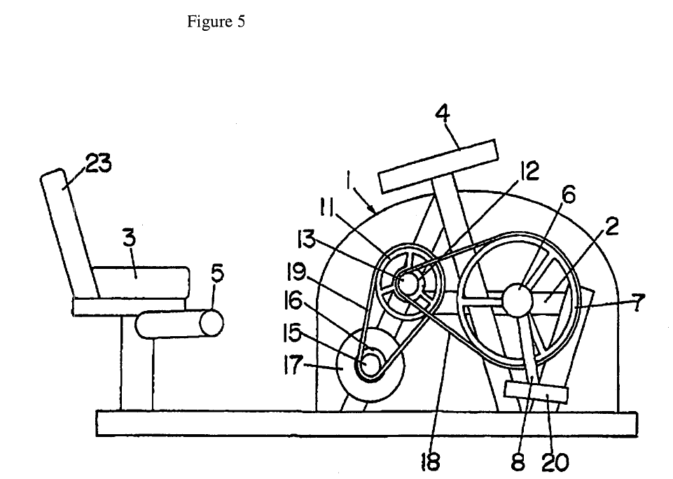

FIG. 5 shows another embodiment. The basic configuration of this embodiment is the embodiment described above, however, and shared parts are labeled by the same reference numbers and their explanation are not repeated.

If it is necessary to apply a load greater than the weight of the subject (especially for young people or athletes) in the cycle-type ergometer 1, this can cause the body to lift up, making it impossible to apply the load. For this reason, when the ergometer is made a recumbent type chair type as shown in FIG. 5, a backrest 23 can be used to resist the load, making it possible to evaluate myodynamia without difficulty even when applying a load greater than the weight of the subject.

The invention is a cycle-type ergometer having a torque measuring means for measuring the torque produced by a pedal and a rotational speed measuring means for measuring the pedal rotational speed, and has a control means for controlling the pedal load such that it gradually increases, measuring the correlation between the torque achieved by a subject and the pedal rotational speed at two or more points until the subject can no longer resist the pedal load, approximating these measurement data by a curve, and estimating the maximum torque achievable by the subject to be the torque achieved by the subject as the pedal rotational speed approaches zero based on this measurement data curve. As a result, measurement data for achieved torque and pedal rotational speed are measured several times until the subject can no longer resist the pedal load and approximated by a curve, and the maximum torque achievable by the subject is estimated based on such a measurement data curve. Therefore, this has the advantage that the maximum torque achievable by the subject can be known by a trial at, for example, 50% or less of the maximum myodynamia even without achieving maximum myodynamia, elevation of the heart rate and blood pressure of the subject can be minimized, the load on bones can be reduced, and myodynamia can be evaluated safely even in the elderly.

The invention has a control means for detecting when the pedal rotational speed measured by the above-mentioned rotational speed measuring means reaches saturation when the subject is asked to pedal as hard as possible under a light load before raising the pedal load, and beginning to increase the pedal load based on this detection result. That is, because the load is increased after the rotational speed becomes saturated, reliable and high-precision data can be sampled repeatedly, and it can be confirmed that the subject has reached the maximum speed at this load. Therefore, this has the advantage that the subject can see clearly the difference made by even a slight change in myodynamia due to the effect of daily training, and this becomes an incentive for daily training.

The invention exercises control such that the pedal load increases in stages. As a result, it is difficult to confirm whether or not the rotational speed is saturated when the pedal load is varied continuously, which can easily lead to errors in measurement data, but by increasing the load in stages, this has the advantage that it can be observed when the rotational speed has become saturated, and the precision of measurement data can be improved.

The invention controls the increment increasing the load in stages to a lower increment if the pedal rotational speed measured by the above-mentioned rotational speed measuring means fell by a greater increment the last time and a greater increment if the pedal rotational speed fell by a lesser increment the last time when comparing the last time to the time before last, and keeps the range of fall in rotational speed within a specific range. That is, the increment increasing the load in stages must be adjusted in various ways according to the myodynamic state of the subject, but when applying a load in stages and the resulting change in speed are observed without a technique for estimating the myodynamia of the subject at the start of pedaling, there may be too little adjustment in the increment increasing the load for persons who find the change in load too great and too much adjustment for persons who find the change in load too little. As a result, this has the advantage that it avoids the number of measurement data being too few for persons lacking strength resulting in a drop in myodynamia estimation precision, or measurement taking too long for persons with good strength resulting in a drop in myodynamia estimation precision due to fatigue.

The invention has a pedal crank angle measuring means for measuring the crank angle of the pedal from a reference position during one revolution and has the data of the subject to be sampled be the torque achieved at the same pedal crank angle during one revolution and the pedal rotational speed, and the invention has the data of the subject to be sampled be the torque achieved at the maximum torque during one revolution and the pedal rotational speed at this time. That is, the timing for sampling data after the rotational speed becomes saturated is not whenever desired, and the torque during one revolution is varied. As a result, this has the advantage that data can be sampled with consistent conditions and good precision by sampling data always at the same pedal crank angle or sampling data at the maximum torque during one revolution.

The invention bases the curve approximating the measurement data on the Hill formula. As a result, the approximation curve may have a certain degree of basis when finding the estimated maximum torque. The Hill formula expresses human myodynamic force and speed characteristics. Because torque is inversely correlated with myodynamia when the pedal crank angle is constant, a hill formula is considered to have an effect improving precision when applied in this case. In addition, results of experiments by the present inventors were close to linear. Therefore, this has the advantage that an easy linear approximation can reduce the number of measurement data, speed up measurement time by the device, and reduce the load on the subject.

The invention estimates the maximum rotational speed as the speed when the load in the approximation curve reaches zero. As a result, the estimated maximum rotational speed is found for rotational speed in the same way as for myodynamia. Therefore, the subject can evaluate not only strength, but also quickness of action. This has the advantage that this is the more important evaluation criterion for persons engaged in sports emphasizing speed more than strength.

The invention creates a general correlation between age and maximum myodynamia statistically from the age and maximum achieved torque of many subjects and finds the maximum myodynamia of a new subject by referring to the muscle-torque age in the above-mentioned correlation chart, and the invention creates a general correlation between age and maximum rotational speed statistically from the age and maximum rotational speed of many subjects and finds the maximum rotational speed of a new subject by referring to the rotational-speed age in the above-mentioned correlation chart. As a result, this has the advantage that subjects who do not engage in sports can evaluate whether their own myodynamia or quickness is good or bad for their age when compared to other people, which can help raise their QOL (quality of life).

Furthermore, because it is not uncommon to find a correlation with physique or weight when comparing to other people, especially when comparing lower limb myodynamia, weight or physique may be normalized to a reference physique to eliminate these factors. Going further, this has the advantage that evaluation reliability can be increased by normalizing body height or leg length to a reference.

In this invention, if it is necessary to apply a load greater than the weight of the subject (especially for young people or athletes), this can cause the body to lift up, making it impossible to apply the load. For this reason, when the ergometer is made a recumbent type=chair type (claim 21), the backrest can be used to resist the load, which has the advantage that myodynamia can be evaluated without difficulty even when applying a load greater than the weight of the subject.

What is claimed is:

1. Device for measuring myodynamia by cycle-type ergometer, characterized in a cycle-type ergometer having a torque measuring means for measuring the torque produced by a pedal and a rotational speed measuring means for measuring the pedal rotational speed, by having a control means for controlling the pedal load such that it gradually increases, measuring the correlation between the torque achieved by a subject and the pedal rotational speed at two or more points until the subject can no longer resist the pedal load, approximating these measurement data by a curve, and estimating the maximum torque achievable by the subject to be the torque achieved by the subject as the pedal rotational speed approaches zero based on this measurement data curve.

2. Method for measuring myodynamia by cycle-type ergometer, characterized in a cycle-type ergometer having a torque measuring means for measuring the torque produced by a pedal and a rotational speed measuring means for measuring the pedal rotational speed, by controlling the pedal load such that it gradually increases, measuring the correlation between the torque achieved by a subject and the pedal rotational speed at two or more points until the subject can no longer resist the pedal load, approximating these measurement data by a curve, and estimating the maximum torque achievable by the subject to be the torque achieved by the subject as the pedal rotational speed approaches zero based on this measurement data curve.

3. Device for measuring myodynamia by cycle-type ergometer described in claim 1, characterized by having a control means for detecting when the pedal rotational speed measured by the above-mentioned rotational speed measuring means reaches saturation when the subject is asked to pedal as hard as possible under a light load before raising the pedal load, and beginning to increase the pedal load based on this detection result.

4. Method for measuring myodynamia by cycle-type ergometer described in claim 2, characterized by detecting when the pedal rotational speed measured by the above-mentioned rotational speed measuring means reaches saturation when the subject is asked to pedal as hard as possible under a light load before raising the pedal load, and beginning to increase the pedal load based on this detection result.

5. Device for measuring myodynamia by cycle-type ergometer described in claim 1 or 3, characterized by exercising control such that the pedal load increases in stages.
